(19) United States
(12) Patent Application Publication
(10) Pub. No.: US 2009/0017122 A1
(43) Pub. Date: Jan. 15, 2009

(54) Drug Forms Having Controlled Bioavailability

(75) Inventors: Peter Serno, Bergisch Gladbach (DE); Roland Heinig, Wuppertal (DE); Kerstin Pauli, Dortmund (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer HealthCare AG, Leverkusen (DE)

(21) Appl. No.: 11/885,061

(22) PCT Filed: Feb. 22, 2006
(86) PCT No.: PCT/EP06/01573

§ 371 Date: Aug. 28, 2008

(30) Foreign Application Priority Data
Mar. 1, 2005 (DE) 102005009241.1

(51) Int. Cl. A61K 9/14; A61K 31/4985

(52) U.S. Cl. 424/489; 514/250

(57) ABSTRACT

The present application relates to novel drug formulations of vardenafil which dissolve rapidly in the mouth and have controlled bioavailability, and to processes for their preparation.

The present application relates to novel drug formulations of vardenafil which dissolve rapidly in the mouth and have controlled bioavailability, and to processes for their preparation.

Imidazotriazinone derivatives such as vardenafil and its use as cGMP phosphodiesterase inhibitors and its activity spectrum are known (for example WO 99/24433), and the compound is commercially available under the name Levitra®. Vardenafil hydrochloride can be administered orally, it being possible to use various oral administration forms such as, for example, tablets, hard gelatin capsules, soft gelatin capsules, powders, granules, chewing tablets or effervescent tablets. A further alternative for administration are administration forms which rapidly disintegrate in the mouth. The patient can take these administration forms quickly, discreetly and without any liquid. In general, these drug forms disintegrate in the mouth in less than 3 minutes, preferably less than 1 minute, and the solution or suspension formed is then swallowed. Accordingly, administration forms which disintegrate rapidly in the mouth are very particularly suitable for patients who have problems swallowing tablets.

Methods for preparing administration forms which disintegrate rapidly in the mouth are known in general. Examples are wafers prepared by freeze-drying, as described in WO 93/23017; the compaction of pulverulent mixtures to rapidly disintegrating tablets, as described in WO 03/051338; the incorporation of the active compounds into films, as described in WO 00/42992.

One problem encountered in the formulation of administration forms which rapidly disintegrate in the mouth is frequently the bitter or otherwise unacceptable taste of the active compound. In these cases, it is possible to prevent completely a dissolution of the active compound in the mouth after the disintegration of the tablet, for example by coating the active compound, active compound granules or coated active compound pellets with polymers which are insoluble in saliva but soluble in gastric juice. In the case of vardenafil and its salts, such as vardenafil hydrochloride trihydrate, this problem is not encountered. The taste of the active compound is only slightly bitter and can easily be masked by adding customary flavors or be integrated into a pleasant taste sensation.

However, it has now been found that other problems do occur with such administration forms prepared by known processes from the active compound vardenafil hydrochloride trihydrate. Following administration of such administration forms, plasma concentration curves are observed in man which differ from those obtained after administration of a commercial vardenafil hydrochloride trihydrate tablet (Levitra®). In particular, there are higher maximum plasma concentrations, and the biological availability of the active compound is higher than after administration of the commercial tablet. In the case of patients which have already undergone prolonged treatment with commercial vardenafil hydrochloride trihydrate tablets for swallowing and which are changed to tablets rapidly disintegrating in the mouth or in the case of patients which, depending on their current circumstances, frequently alternate between tablets for swallowing and tablets which disintegrate rapidly in the mouth, this may be undesirable. Surprisingly, we have found vardenafil formulations which rapidly disintegrate in the mouth where these unexpected and undesirable changes of the pharmacokinetic profile are avoided. Using these formulations according to the invention, the patient can benefit from the advantage of drug forms which rapidly disintegrate in the mouth, such as the fact that they can be swallowed easily or be taken without liquid, without the disadvantages described.

To this end, in accordance with the present invention, it is necessary for the release rate of vardenafil after administration of the administration form which disintegrates rapidly in the mouth to be limited. Furthermore, it has been found that this limitation of active compound release can be determined by measuring the dissolution rate of vardenafil in the USP paddle stirrer apparatus in physiological saline at 37° C. and 50 rotations per minute, and that, using this determination method, it is possible to define a release rate for the administration form according to the invention where within the first 5 minutes not more than 50% of the dose may go into solution.

For preparing formulations which satisfy the dissolution rate criterion according to the invention, a number of different processes, described below, were found.

One alternative for preparing the drug formulation according to the invention is to use vardenafil in the form of vardenafil dihydrate or anhydrous vardenafil.

According to one of the known processes for preparing drug forms which disintegrate rapidly in the mouth, the active compound processed is vardenafil dihydrate or anhydrous vardenafil. Here, drug forms which disintegrate rapidly in the mouth are to be understood as meaning drug forms whose disintegration time (method of the European pharmacopoea) is less than 3 minutes, preferably less than 1 minute.

These drug forms are formed by mixing the active compound with sugars, sugar alcohols, disintegrants or other disintegration-enhancing substances and also further auxiliaries, such as surfactants, lubricants, flow regulators, flavors, colorants or fillers, and compression on a tableting machine. Alternatively, the anhydrous vardenafil or the vardenafil dihydrate can be dissolved or suspended together with auxiliaries such as sugar alcohols, polymers or surfactants in an aqueous solvent, and the solution or suspension is metered into blister wells and subjected to a freeze-drying process. As another alternative, the anhydrous vardenafil or the vardenafil dihydrate can be dissolved or suspended together with auxiliaries such as film-formers, plasticizers, flavors and colorants in an organic solvent and then be processed to a film. A solvent-free film production using meltable film formulations is also possible. After preparation, the films are cut into pieces corresponding to individual doses.

Another alternative to achieve the low dissolution rate according to the invention of vardenafil in physiological saline is the use of a vardenafil salt having a low solubility in water. By adding an additive comprising the same ions, the solubility of these salts and thus the dissolution rate, too, can be reduced even further, if required.

A further alternative to achieve the low dissolution rate according to the invention of vardenafil in physiological saline is the prior treatment of a water-soluble vardenafil salt such that the release rate according to the invention is obtained. Suitable for this purpose is in particular coating the active compound salts with polymer(s) or embedding them in polymer(s). Here, the vardenafil salts may be solvent-free or solvent-containing and may be present in various polymorphic forms. Examples of water-soluble salts are vardenafil hydrochloride trihydrate, vardenafil dimesylate monohydrate and vardenafil monomesilate. However, salts of vardenafil with citric acid, tartaric acid, succinic acid, sulfuric acid, acetic acid, adipic acid, gluconic acid, glucuronic acid, glutamic acid, glutaric acid, glycerolphosphoric acid, lactic acid, maleic acid, malic acid, phosphoric acid, lactobionic acid, malonic acid, naphthalenesulfonic acid, naphthalenedisulfonic acid or toluenesulfonic acid are also possible. Alternatively, it is also possible to obtain the water-soluble vardenafil salt(s) by joint processing of vardenafil and acid in the drug form. In this case, the corresponding salt is formed in the mouth following access of aqueous medium. The release rate according to the invention by coating with or embedding in polymers can be achieved in a pH-controlled or time-controlled manner. Suitable for the pH-controlled release are physiologically acceptable polymers which are insoluble at neutral pH and soluble at acidic pH, and in particular basic butyl methacrylate copolymer (for example Eudragit® E 100). The time-controlled release is achieved by coating with or embedding in physiologically acceptable polymers, by way of example and by way of preference with ethylcellulose. Initially, these polymers limit the release of active compound to the rate according to the invention, but subsequently the active compound is released by diffusion or tearing of the film.

For coating the active compound with polymer, active compound crystals, granules or pellets are coated in a suitable apparatus and according to suitable processes, such as in a fluidized bed or in a coating drum, with polymer solution or polymer melt. Coating by spray drying or spray solidification is also possible. Coating of the active compound may also be achieved in a coazervation process. For embedding the active compound, the active compound is compressed jointly with the polymer on a roll or in a tableting machine. The joint precipitation of active compound and polymer in a coprecipitation process is likewise possible. For processing the preferred polymer poly(butylmethacrylate-co-(2-dimethylamino-ethyl)methacrylate-co-methylmethacrylate) (Eudragit® E 100), the polymer is either dissolved in acetone/isopropanol/water, or an aqueous dispersion of the finely ground substance is prepared which, in addition to polymer and water, also comprises surfactants, such as sodium lauryl sulfate, and release agents, such as magnesium stearate. The solution or dispersion obtained in this manner is sprayed onto active compound-comprising particles, for example granules. Suitable for this purpose is a fluidized bed process, for example coating in a Wurster tube or spraying-on in coaters. Here, a typical application rate is, for example, 1 mg of polymer per cm² of particle surface.

A further alternative to achieve the low dissolution rate according to the invention in the physiological saline is the use of coarse particle size fractions of a vardenafil salt, for example of vardenafil hydrochloride trihydrate, in particular of vardenafil hydrochloride trihydrate having a mean particle size >80 μm. To reduce the dissolution rate even further, the particles may also be coated.

After pretreatment of the vardenafil salt in one of the processes described such that the release rate according to the invention can be achieved, the salt is processed as active compound according to one of the known processes for preparing drug forms which rapidly disintegrate in the mouth.

Suitable for this purpose is the mixing of the pretreated active compound with sugars, sugar alcohols, disintegrants or other disintegration-enhancing substances and also further auxiliaries, such as surfactants, lubricants, flow regulators, flavors, colorants or fillers, and compression on a tableting machine. Alternatively, the pretreated vardenafil salt can be dissolved or suspended together with auxiliaries such as sugar alcohols, polymers or surfactants in an aqueous solvent, and the suspension is metered into blister wells and subjected to a freeze-drying process. As another alternative, the pretreated vardenafil salt can be suspended together with auxiliaries such as film-formers, plasticizers, flavors and colorants in an organic solvent and then be processed to a film. A solvent-free film production using meltable film formulations is also possible. After preparation, the films are cut into pieces corresponding to individual doses.

### COMPARATIVE EXAMPLE 1

### Excess Bioavailability and Increased Maximum Plasma Concentration after Administration of Non-Inventive Tablets which Rapidly Disintegrate in the Mouth

Tablets comprising 23.7 mg of vardenafil hydrochloride trihydrate, 0.748 mg of yellow iron oxide, 0.102 mg of red iron oxide, 1.02 mg of apricot flavor, 0.17 mg of neohesperidine dihydrochalcone, 3.40 mg of aspartam, 0.850 mg of finely divided silica, 4.25 mg of magnesium stearate and 135.76 mg of Pharmaburst® (commercial mixture from SPI) are prepared by mixing all components and compressing them directly on a rotary tableting machine. The release of active compound in 900 ml of physiological saline at 37° C. and 50 rotations per minute in the USP paddle stirrer apparatus is 85% in 5 minutes. Thus, the solvent rate criterion according to the invention is not met. For comparison, in a crossover experiment, a tablet to be swallowed with water and comprising the following components: 23.705 mg of vardenafil hydrochloride trihydrate (corresponds to 20 mg of vardenafil), 141.797 mg of microcrystalline cellulose, 8.85 mg of crosslinked polyvinylpyrrolidone, 0.885 mg of colloidal silica, 1.770 mg of magnesium stearate, 3.385 mg of hypromellose, 1.128 mg of Macrogol 400, 0.925 mg of titanium dioxide, 0.188 mg of yellow iron oxide and 0.015 mg of red iron oxide is administered to twelve subjects. After administration of the commercial tablet for swallowing (Levitra®), a maximum plasma concentration of 20.1 μg/l (geometric mean) is obtained, after administration of the non-inventive tablet of this comparative example, which rapidly disintegrates in the mouth, a maximum plasma concentration of 25.1 μg/l (geometric mean) is obtained. The relative bioavailability of the non-inventive tablet which disintegrates in the mouth is 128%.

### EXAMPLE 2

### Demonstration of Approximately Corresponding Bioavailability of an Inventive Tablet which Rapidly Disintegrates in the Mouth with a Tablet for Swallowing

A tablet which disintegrates in the mouth, comprising 10.7 mg of vardenafil dihydrate (corresponds to 10 mg of vardenafil), 0.484 mg of yellow iron oxide, 0.066 mg of red iron oxide, 1.1 mg of apricot flavor, 4.4 mg of aspartam, 6.6 mg of magnesium stearate and 196.65 mg of Pharmaburst® B2 (commercial auxiliary mixture from SPI) is prepared by mixing vardenafil dihydrate, yellow iron oxide, red iron oxide, apricot flavor, aspartam and Pharmaburst® in a compulsory mixer and then mixing this mixture with magnesium stearate in a tumbling mixer. This tablet which rapidly disintegrates in the mouth is in accordance with the invention since in physiological saline at 37° C. over a period of 5 minutes less than 9% of the dose goes into solution, and the dissolution rate criterion according to the invention is thus met. In a crossover comparison with 11 subjects, the relative bioavailability is examined in comparison to a reference tablet of the following composition: 11.852 mg of vardenafil hydrochloride trihydrate (corresponds to 10 mg of vardenafil), 105.023 mg of microcrystalline cellulose, 6.25 mg of crosslinked polyvinylpyrrolidone, 0.625 mg of colloidal silica, 1.25 mg of magnesium stearate, 2.391 mg of hypromellose, 0.797 mg of Macrogol 400, 0.653 mg of titanium dioxide, 0.133 mg of yellow iron oxide and 0.011 mg of red iron oxide. As a measure for the bioavailability of the test formulations, the area under the plasma concentration/time curve (AUC) was used, which was 34.9 μg*h/l for the tablet according to the invention and 35.7 μg*h/l for the reference tablet (in each case the geometric mean). The maximum plasma concentration of the tablet according to the invention could be limited to 79% of that of the reference tablet.

### EXAMPLE 3

### Demonstration of Approximately Corresponding Bioavailability of an Inventive Tablet which Rapidly Disintegrates in the Mouth with a Tablet for Swallowing

In each case one tablet which disintegrates in the mouth and comprises 10.7 mg of vardenafil dihydrate (corresponds to 10 mg of vardenafil), 5 mg of ground succinic acid, 0.484 mg of yellow iron oxide, 0.066 mg of red iron oxide, 1.1 mg of apricot flavor, 4.4 mg of aspartam, 6.6 mg of magnesium stearate and 191.65 mg of Pharmaburst® B2 (commercial auxiliary mixture from SPI) is administered to 11 subjects. This tablet which rapidly disintegrates in the mouth is in accordance with the invention, since the release of active compound in 900 ml of physiological saline at 37° C. and 50 rotations per minute in the USP paddle stirrer apparatus is only 40% over a period of 5 minutes, and the dissolution rate criterion according to the invention is thus met. In a crossover comparison with the reference tablet described in comparative example 2, the relative bioavailability is 101.8%.

### EXAMPLE 4

### Demonstration of Approximately Corresponding Bioavailability of an Inventive Tablet which Rapidly Disintegrates in the Mouth with a Tablet for Swallowing

118 g of anhydrous vardenafil, 590 g of manitol and 11.8 g of Poloxamer 188 are suspended or dissolved in 2360 g of water. In a fluidized bed apparatus, the suspension is sprayed onto 848 g of Pharmaburst® B2 (commercial auxiliary mixture from SPI) and 44.8 g of aspartam. The granules are dried in the fluidized bed and subsequently mixed with 2714 g of Pharmaburst® B2, 22.42 g of pulverulent orange flavor and 134.5 g of magnesium stearate. On a rotary tablet press, this mixture is compressed to round tablets having a diameter of 11 mm and a mass of 380 mg. The release rate of this tablet which rapidly disintegrates in the mouth is 49% over 5 minutes at 37° C. In a crossover comparison with 11 healthy subjects, the pharmacokinetics of this tablet which disintegrates in the mouth are tested against the reference tablet described in example 2. A mean relative bioavailability of 92% and a mean maximum plasma concentration of 84% of the reference tablet are found.

### EXAMPLE 5

Poly(butylmethacrylate-co-(2-dimethylamoethyl)methacrylate-co-methylmethacrylate) (Eudragit® E 100) is ground on a fluidized-bed counterjet mill. 152.07 g of the ground product are mixed with 47.93 g of micronized vardenafil hydrochloride trihydrate, sieved and mixed again. The mixture is compacted on a roll and comminuted via a 1 mm sieve. 14.31 g of the granules obtained in this manner are mixed with 0.55 g of pulverulent orange flavor, 1.1 g of aspartam, 90.74 g of Pharnaburst® B2 and 3.3 g of magnesium stearate and compressed to tablets of a diameter of 11 mm and a mass of 380 mg. In the USP paddle stirrer apparatus, in 900 ml of physiological saline at 37° C. and 50 rotations per minute, the tablets obtained in this manner, which rapidly disintegrate in the mouth, release 42% of the active compound over a period of 5 minutes.

### EXAMPLE 6

9 parts of poly(butylmethacrylate-co-(2-dimethylaminoethyl)methacrylate-co-methylmethacrylate) (Eudragit® E 100) are dissolved in 60 parts of isopropanol and 4 parts of water. 27 parts of micronized vardenafil hydrochloride trihydrate are suspended in this solution. The suspension is evaporated to dryness and the residue is removed, ground and sieved. 4.16 g of the coprecipitate obtained in this manner are mixed with 5 g of a colorant premix comprising 95 parts of Pharmaburst® B2, 4.4 parts of yellow iron oxide and 0.6 part of red iron oxide, 0.5 g of pulverulent orange flavor, 1 g of aspartam, 86.34 g of Pharmaburst® B2 and 3 g of magnesium stearate and compressed to tablets having a diameter of 11 mm and a mass of 380 mg. In the USP paddle stirrer apparatus, in 900 ml of physiological saline at 37° C. and 50 rotations per minute, the tablets obtained in this manner, which rapidly disintegrate in the mouth, release 47% of the active compound over a period of 5 minutes.

### EXAMPLE 7

11.85 g of vardenafil hydrochloride trihydrate having a mean particle size of 135 μm are mixed with 1 g of magnesium stearate for 30 minutes, sieved through a 0.8 mm sieve and mixed again for another 30 minutes. 5 g of colorant premix comprising 95 parts of Pharmaburst® B2, 4.4 parts of yellow iron oxide and 0.6 part of red iron oxide, 0.5 g of pulverulent orange flavor, 1 g of aspartam, 77.65 g of Pharmaburst® B2 and 3 g of magnesium stearate are then added and mixed. On a tableting machine, the mixture ready for compression is compressed to round tablets having a diameter of 7 mm and a mass of 100 mg. In the USP paddle stirrer apparatus, in 900 ml of physiological saline at 37° C. and 50 rotations per minute, the tablets obtained in this manner, which rapidly disintegrate in the mouth, release 49% of the active compound over a period of 5 minutes.

### EXAMPLE 8

2.44 g of micronized vardenafil dihydrate, 0.68 g of tartaric acid which had been pulverized and sieved through a 0.35 mm sieve and 45.37 g of Pharmaburst® B2 are mixed for 5 minutes, sieved through a 0.5 mm sieve and mixed again for 5 minutes. 1.5 g of magnesium stearate are added, and the mixture is mixed for another 5 minutes. On a tableting machine, this mixture is compressed to round tablets having a diameter of 9 mm and a mass of 220 mg. In the USP paddle stirrer apparatus, in 900 ml of physiological saline at 37° C. and 50 rotations per minute, the tablets obtained in this manner, which rapidly disintegrate in the mouth, release 45% of the active compound over a period of 5 minutes.

### EXAMPLE 9

10.0 g of anhydrous vardenafil base, 7.6 g of crospovidone, 38 g of calcium silicate, 57 g of microcrystalline cellulose, 246.5 g of spray-dried manitol, 3.8 g of aspartam and 1.9 g of pulverulent orange flavor are mixed and subjected to dry granulation. The granules are subsequently mixed with 3.8 g of finely divided silica and 11.4 g of magnesium stearate and compressed to tablets having a diameter of 11 mm and a mass of 380 mg.

### EXAMPLE 10

130 g of anhydrous vardenafil are mixed with 24.7 g of orange flavor, 49.4 g of aspartam and 4563 g of Pharmaburst® B2 (commercial auxiliary mixture from SPI), the mixture is sieved through a 0.5 mm sieve, mixed again and subjected to dry granulation on a roll. 24.7 g of finely divided silica and 148.2 g of magnesium stearate are added to the granules, and the mixture is mixed in a tumble mixer for 5 minutes. On a tableting machine, the mixture is compressed to tablets having a mass of 380 mg. The tablets, which rapidly disintegrate in the mouth, meet the dissolution rate criterion according to the invention since only about 26% of the administered dose are released in 900 ml of physiological saline at 37° C. over a period of 5 minutes.

### EXAMPLE 11

107 g of anhydrous vardenafil and 536 g of erythritol are dissolved in 10.7 kg of 80% ethanol and, in a vacuum fluidized-bed apparatus, sprayed onto 1.5 kg of Pharmaburst® B2. The solid vardenafil solution prepared in this manner is subsequently mixed with 20.4 g of pulverulent orange flavor, 40.71 g of aspartam, 1745.4 g of Pharmaburst® B2 and 122.1 g of magnesium stearate and, on a rotary tablet press having plasma-chromed punches, compressed to tablets having a diameter of 11 mm and a mass of 380 mg.

1. A drug formulation which disintegrates rapidly in the mouth and comprises vardenafil and has controlled bioavailability, characterized in that the active compound released after 5 minutes in the USP paddle stirrer apparatus at 50 revolutions per minute in physiological saline at 37° C. is less than 50% of the vardenafil dose present in the formulation.

2. The drug formulation as claimed in claim 1, comprising vardenafil dihydrate or anhydrous vardenafil base.

3. The drug formulation as claimed in claim 1, comprising vardenafil hydrochloride trihydrate having a mean particle size of more than 80 μm.

4. The drug formulation as claimed in claim 1, comprising a vardenafil salt with a physiologically acceptable acid or a mixture of vardenafil and a physiologically acceptable acid, characterized in that the active compound and/or the acid are/is coated with a polymer or embedded into a polymer matrix.

5. The drug formulation as claimed in claim 4, comprising a vardenafil salt with a physiologically acceptable acid or a mixture of vardenafil and a physiologically acceptable acid, characterized in that the active compound and/or the acid are/is coated with a polymer or embedded into a polymer matrix, the polymer being insoluble in saliva and soluble in gastric juice.

6. The drug formulation as claimed in claim 5, where the polymer is poly(butylmethacrylate-co-(2-dimethylaminoethyl)methacrylate-co-methylmethacrylate) (Eudragit® E 100 or Eudragit® E PO).

7. The drug formulation as claimed in claim 4, where the polymer is ethylcellulose.
